(19) United States
(12) Patent Application Publication
(10) Pub. No.: US 2026/0191846 A1
(43) Pub. Date: Jul. 9, 2026

(54) ORAL IMMEDIATE-RELEASE FORMULATION COMPRISING ILAPRAZOLE WITH IMPROVED STABILITY AS ACTIVE INGREDIENT AND METHOD FOR PREPARING SAME

(71) Applicant: KOOKMIN UNIVERSITY INDUSTRY ACADEMY COOPERATION FOUNDATION, Seoul (KR)

(72) Inventor: Joo-Eun KIM, Seoul (KR)

(73) Assignee: KOOKMIN UNIVERSITY INDUSTRY ACADEMY COOPERATION FOUNDATION, Seoul (KR)

(21) Appl. No.: 19/133,273

(22) PCT Filed: Sep. 21, 2023
(86) PCT No.: PCT/KR2023/014383

§ 371 Date: May 28, 2025

(30) Foreign Application Priority Data
Nov. 28, 2022 (KR) 10-2022-0161328

(51) Int. Cl. A61K 31/4439; A61K 9/00; A61K 9/20; A61K 9/24; A61K 9/28; A61K 33/00

(52) U.S. Cl. CPC A61K 31/4439; A61K 9/0053; A61K 9/2009; A61K 9/2013; A61K 9/2018; A61K 9/2054; A61K 9/2059; A61K 9/209; A61K 9/2866; A61K 33/00

(57) ABSTRACT

The present invention relates to an oral immediate-release formulation comprising ilaprazole with improved stability as an active ingredient and a method for preparing same, wherein the present invention relates to an oral immediate-release formulation and a method for preparing same, the oral immediate-release formulation comprising ilaprazole and a stabilizer in an inner core portion thereof and an antacid in an outer layer portion thereof, in which the oral immediate-release formulation has improved compounding stability by suppressing contact between the inner core portion and the outer layer portion through coating on the inner core portion, has excellent stability by minimizing the amount of related substances generated, and has a fast drug effect (the time to reach maximum blood concentration (Tₘₐₓ) ranges from 0.3 hours to 0.8 hours) and excellent stability, through the rapid antacid effect of the antacid.

### TECHNICAL FIELD

The present disclosure relates to an oral immediate-release formulation including ilaprazole with improved stability as an active ingredient and a preparation method thereof. Specifically, the present disclosure relates to an oral immediate-release formulation and a preparation method thereof, wherein, in order to improve stability of ilaprazole which is highly unstable under an acidic condition, the oral immediate-release formulation contains a stabilizer used in an inner core and an antacid in an outer layer so as to suppress decomposition of an active ingredient in gastric acid and thus maintain a storage stability of the formulation for a long period of time even under accelerated storage conditions, and it is also possible to allow, when administered orally, ilaprazole in the inner core to be rapidly absorbed in the stomach by neutralization of gastric acid by the antacid in the outer layer in the stomach.

### BACKGROUND ART

Ilaprazole is a derivative of 5-pyrrolyl-2-pyridyl methyl sulfonyl benzimidazole that suppresses gastric acid secretion by inhibiting H⁺/K⁺ ATPase on an acid-secreting surface of parietal cells in the gastric mucosa. In general, proton pump inhibitors (PPIs) are recommended as an effective first-line therapy, providing higher gastric acid suppression with fewer side effects than histamine H2 receptor blockers. Ilaprazole is effective in the treatment of gastric and duodenal ulcers, esophageal reflux disease, and Helicobacter pylori infection by inducing dose-dependent suppression of basal acid secretion and stimulation-induced acid secretion.

While most PPI agents have a probability of having drug interactions due to the influence of genetic diversity on CYP, an enzyme involved in drug metabolism, ilaprazole is known to be metabolized mostly through non-enzymatic systems. In addition, ilaprazole has an elimination half-life of approximately 10 hours, which is known to be the longest half-life among PPI agents, making it effective in alleviating symptoms of nocturnal acid secretion (NAB).

Meanwhile, it is a well-known fact in the art that PPI agents are easily decomposed or transformed in acidic and neutral media. Various PPI agents, such as ilaprazole, rabeprazole or esomeprazole, omeprazole, and pantoprazole, are very unstable under acidic conditions, such that to prevent rapid decomposition in acidic gastric juice when administered orally and ensure that the active ingredients reach the small intestine, formulations in the form of enteric coated tablets or enteric capsules are used. However, this enteric coating design method is designed to allow polymers used in the enteric coating to be dissolved and absorbed in the intestine rather than being immediately absorbed in the stomach, which results in a slow on-set time and thus is not suitable for gastrointestinal diseases that require immediate therapeutic effects.

Therefore, stable conditions are required to manufacture ilaprazole tablets for rapid treatment effects for gastric ulcer. There have been no reports to date on immediate-release formulations as a tablet containing ilaprazole as an active ingredient, rather than enteric coated tablets.

Under these circumstances, the inventors of the present disclosure conducted research to develop a formulation that secures a long-term pharmaceutical stability of ilaprazole tablets, suppresses loss of activity due to gastric acid when administered orally, and is capable of facilitating absorption of the drug in the stomach or upper duodenum. Through the study above, the inventors of the present disclosure discovered that the stability of ilaprazole tablets is greatly affected by the type of stabilizers.

Therefore, the inventors of the present disclosure sought to develop an oral immediate-release formulation that has excellent physicochemical stability for a long period of time and is able to show rapid absorption and efficacy of ilaprazole in the stomach and upper duodenum.

### DISCLOSURE OF THE INVENTION

### Technical Goals

In order to address issues regarding accelerated decomposition of ilaprazole by low pH, heat, or moisture as well as limited absorption of ilaprazole in the stomach due to its physicochemical properties of being sensitive in terms of stability while enteric coating is essentially required, an object of the present disclosure is to provide an oral immediate-release formulation which ensures excellent physical stability for a long period of time and exhibits rapid absorption and efficacy of ilaprazole in the stomach and the upper duodenum by having a stabilizer and an appropriate coating agent in the oral formulation containing ilaprazole and an antacid simultaneously.

### Technical Solutions

In order to achieve the above object, the present disclosure provides an oral immediate-release formulation which includes an inner core including ilaprazole as an active ingredient and a stabilizer, and an outer layer surrounding the inner core and including an antacid.

In addition, the present disclosure provides an oral immediate-release formulation which includes: an inner core including a tablet including ilaprazole as an active ingredient and calcium hydroxide as a stabilizer, and a polymer-containing coating layer which coats the tablet; and an outer layer surrounding the inner core and including sodium bicarbonate as an antacid.

Additionally, the present disclosure provides a method of preparing an oral immediate-release formulation, including (i) mixing ilaprazole, a stabilizer, and a pharmaceutically acceptable additive to prepare a mixture (step 1); (ii) first compressing the mixture to prepare an inner core in the form of a tablet (step 2); and (iii) second compressing a mixture obtained by mixing an antacid and a pharmaceutically acceptable additive on the inner core to prepare an outer layer in the form of a tablet (step 3).

### Advantageous Effects

An oral immediate-release formulation according to the present disclosure prevents ilaprazole from being decomposed by neutralizing the acidic environment in the stomach, as an antacid, such as sodium bicarbonate, is released before ilaprazole, and keeps ilaprazole as a main ingredient from being decomposed for about 30 minutes which is the time for hydroxide salt used as a stabilizer in ilaprazole tablets to disintegrate in gastric acid. Furthermore, in order to secure a long-term stability of a finished drug product of ilaprazole, by containing a stabilizer in the form of a hydroxide salt for ilaprazole in an inner core, it is possible to suppress decomposition of an active ingredient under acidic conditions to provide an effect of retaining a storage stability of a formulation for a long period of time even under accelerated storage conditions.

In particular, when containing one or more of hydroxide salts as the stabilizer, such as calcium hydroxide, magnesium hydroxide, and sodium hydroxide, it is possible to achieve excellent stability as generation of related substances is minimized for a long period of time (for example, about 6 months) even under accelerated storage conditions, and by determining an in-vitro elution profile of an ilaprazole immediate-release formulation by including an antacid that increases the gastric pH to allow rapid absorption in the stomach, it is expected to have effects such as high elution rate and bioavailability of ilaprazole.

Moreover, the oral immediate-release formulation according to the present disclosure not only has excellent physicochemical stability for a long period of time, but also, through a rapid antacid effect of antacids such as sodium carbonate, sodium bicarbonate, calcium carbonate, and potassium carbonate, ilaprazole is immediately eluted and absorbed in the stomach to have a quick acting effect, and it is also possible to provide a PPI formulation and a preparation method thereof to which various immediate-release designs are applied, by utilizing the present disclosure to ilaprazole as well as to other PPIs.

### BEST MODE FOR CARRYING OUT THE INVENTION

The inventors of the present disclosure designed a use of a stabilizer to secure a long-term stability of ilaprazole formulations and a use of an antacid to increase the gastric pH for rapid absorption in the stomach.

In particular, by containing ilaprazole and one or more hydroxide salts such as calcium hydroxide, magnesium hydroxide, and sodium hydroxide as stabilizers in an inner core as well as one of sodium carbonate, sodium bicarbonate, calcium carbonate, and potassium carbonate as an antacid in an outer layer, and coating a polymer-containing thin coating agent on the inner core so as to interfere with the contact between the inner core and the outer layer, it was found that the formulation is physicochemically stable for a long period of time (e.g., about 6 months under accelerated storage conditions) and has a immediate-release elution profile (e.g., 80% or greater within 30 minutes).

Therefore, the inventors of the present disclosure intended to provide an oral immediate-release formulation which may have excellent physicochemical stability for a long period of time and exhibit rapid absorption and efficacy of ilaprazole in the stomach and upper duodenum, using a stabilizer and an appropriate coating agent in the formulation containing ilaprazole and antacid simultaneously.

Hereinafter, the present disclosure will be described in more detail.

The present disclosure provides an oral immediate-release formulation which includes an inner core including ilaprazole as an active ingredient and a stabilizer, and an outer layer surrounding the inner core and including an antacid.

The term “ilaprazole” as used herein may refer to a compound represented by the following Chemical Formula 1, which is “2-[(4-methoxy-3-methylpyridin-2-yl)methanesulfonyl]-5-(1H-pyrrol-1-yl)-1H-1,3-benzoimidazole (generic name ilaprazole)”.

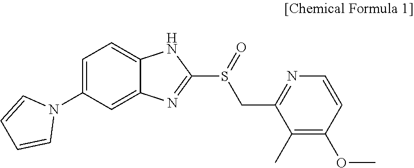 [SMILES: COc1ccnc(CS(=O)c2nc3cc(-n4cccc4)ccc3[nH]2)c1C]

The ilaprazole may be one that is commercially available or directly synthesized. The content of the ilaprazole may be in a therapeutically effective range of 1 mg to 20 mg per formulation, but preferably it may include in an amount of 5 mg to 15 mg.

The stabilizer in the inner core may be one or more stabilizers selected from the group consisting of calcium hydroxide, magnesium hydroxide, sodium hydroxide, potassium hydroxide, meglumine, magnesium oxide, calcium carbonate, sodium bicarbonate, calcium hydrogen phosphate, potassium hydrogen phosphate dihydrate, calcium phosphate tribasic, monoethanolamine, potassium bicarbonate, potassium citrate, sodium borate, sodium citrate dihydrate, and tri ethanol amine.

The stabilizer may be in a range that provides excellent stability of the tablet from a pharmaceutical perspective and preferably include in an amount of 15 to 60 wt % with respect to 100 wt % of the entire inner core, and more preferably 15 to 40 wt %. Since the storage stability and acid stability of the formulation are barely improved when the above range is exceeded, it may be desirable to use it within the above range.

The inner core may include 2 to 5 parts by weight of a stabilizer per 1 part by weight of ilaprazole, and preferably 3 to 4 parts by weight may be used.

In particular, one or more of hydroxide salts, such as calcium hydroxide, magnesium hydroxide, and sodium hydroxide, may be used as a stabilizer in the inner core, where the hydroxide salt plays a role in improving the storage stability and acid stability of the formulation, thereby remarkably suppressing an increase of related substances under room temperature and accelerated storage conditions.

The outer layer including the antacid may surround an upper part of the inner core, and as the antacid, the antacid may be one or more selected from the group consisting of sodium bicarbonate, sodium carbonate, potassium carbonate, sodium glycine carbonate, calcium carbonate, sodium sulfite, sodium bisulfite, sodium metabisulfite, and potassium bicarbonate.

Here, the content of the antacid may be in a therapeutically effective content range including 600 to 1100 mg per formulation, and in particular, when the content of sodium bicarbonate used as the antacid exceeds 800 mg per formulation, it may affect the intake convenience, physical properties, and therapeutic effects for patients with dysphagia.

The oral immediate-release formulation of the present disclosure may further include a coating layer containing a polymer between the inner core and the outer layer.

In addition, the present disclosure provides an oral immediate-release formulation which includes: an inner core including a tablet including ilaprazole as an active ingredient and calcium hydroxide as a stabilizer, and a polymer-containing coating layer which coats the tablet; and an outer layer surrounding the inner core and including sodium bicarbonate as an antacid.

The polymer may be one or more polymers selected from the group including hydroxypropyl methyl cellulose (HPMC), hydroxyethyl methyl cellulose, hydroxypropyl cellulose, hydroxymethyl cellulose, hydroxyethyl cellulose, carboxymethyl cellulose, carboxymethyl cellulose calcium, carboxymethyl cellulose sodium, ethyl cellulose, Eudragit L 100-55, a mixture of Eudragit RS and Eudragit RL, cellulose acetate, cellulose acetate phthalate, hydroxypropyl methyl cellulose acetate succinate, hydroxypropyl methyl cellulose phthalate, polyethyl acetate-methyl acetate copolymer, polyvinyl phthalate acetate, and shellac.

The polymers included in the coating layer may be included in an amount of 0.5 to 5 parts by weight, respectively, with respect to 100 parts by weight of the entire inner core.

In particular, the coating layer may include HPMC as a first polymer and ethyl cellulose as a second polymer and may be to coat an outer surface of the inner core using a first coating solution including HPMC and a second coating solution including ethyl cellulose, and the coating layer may serve to improve a compounding stability by separating and/or inhibiting direct contact between the inner core including ilaprazole and the outer layer including sodium bicarbonate. However, the second polymer is not used for the purpose of achieving the long-acting effect, but rather, for the effect of immediate release and stability of the cored tablet, and it must be coated in an amount of less than 5 parts by weight based on the total weight of the formulation to maintain the role of immediate release and stability of the inner core.

In addition, the second coating solution may be thinly coated in an amount of 2 to 5 parts by weight with respect to 100 parts by weight of the entire inner core to induce a coating film to dissolve after the sodium bicarbonate in the outer layer is eluted and the intragastric pH increases, and prevent ilaprazole from being decomposed by making ilaprazole immediately eluted before the neutralized intragastric pH decreases again after elution of the sodium bicarbonate. The coating layer is not limited to coating of the first polymer and the second polymer in the first and second layers, and even if only a single layer is coated by 5% or less, the same immediate-release pattern effect was exhibited.

The outer layer may further include povidone or copovidone as a binder and further include crospovidone or sodium starch glycolate as a dissolution aid.

When the oral immediate-release formulation of the present disclosure is administered orally, immediately after the antacid effect due to rapid elution of the antacid from the outer layer in the stomach, ilaprazole may be released from the inner core.

When a drug elution test was performed with 900 mL of water (Korean Pharmacopoeia, Elution Test Method) at a rotation rate of 75 rpm and a medium temperature of 37.5° C., the oral immediate-release formulation may have an antacid elution rate of 80% or greater at 15 minutes and an antacid elution rate of 100% or greater at 30 minutes. The antacid may be sodium bicarbonate, and the ion chromatogram may be used as an analyzer.

In addition, when the oral immediate-release formulation is administered to 500 mL of pH 1.2 buffer, and then, after approximately 10 minutes which is the time for the outer layer to almost disintegrate or be eluted, additional elution solution to make 900 mL (final volume of 900 mL) of pH 10.0 buffer (FDA dissolution method DB, Rabeprazole sodium Tablets) is added to conduct a drug elution test at a rotation rate of 100 rpm and a medium temperature of 37.5° C., the elution rate of ilaprazole is 80% or greater at 30 minutes.

Further, the present disclosure provides a method of preparing an oral immediate-release formulation, including (i) mixing ilaprazole, a stabilizer, and a pharmaceutically acceptable additive to prepare a mixture (step 1); (ii) first compressing the mixture to prepare an inner core in the form of a tablet (step 2); and (iii) second compressing a mixture obtained by mixing an antacid and a pharmaceutically acceptable additive on the inner core to prepare an outer layer in the form of a tablet (step 3).

The first step is to prepare a mixture by mixing ilaprazole, the stabilizer, and the pharmaceutically acceptable additive, wherein the stabilizer may be used in an amount of 30 to 60 wt % with respect to 100 wt % of the entire inner core.

The ilaprazole may be included in a therapeutically effective content range of 1 mg to 20 mg per formulation, but preferably 5 mg or 15 mg.

The second step is to prepare the inner core in the form of a tablet by first compressing the mixture, wherein, when preparing the tablet herein, the tablet may be manufactured by a direct compression method, but is not limited thereto.

The third step is to second compress using a mixture obtained by mixing the antacid and the pharmaceutically acceptable additive to coat the inner core prepared in the second step and may further include preparing a coating layer containing a polymer between the second and third steps.

The polymer may be one or more polymers selected from the group consisting of hydroxypropyl methyl cellulose (HPMC), hydroxyethyl methyl cellulose, hydroxypropyl cellulose, hydroxymethyl cellulose, hydroxyethyl cellulose, carboxymethyl cellulose, carboxymethyl cellulose calcium, carboxymethyl cellulose sodium, ethyl cellulose, Eudragit L 100-55, a mixture of Eudragit RS and Eudragit RL, cellulose acetate, ethyl cellulose, cellulose acetate phthalate, hydroxypropyl methyl cellulose acetate succinate, hydroxypropyl methyl cellulose phthalate, polyethyl acetate-methyl acetate copolymer, polyvinyl phthalate acetate, and shellac.

In particular, the coating layer may include HPMC as the first polymer and ethyl cellulose as the second polymer, and a lubricant or a fluidizing agent may further be included as an additive in the coating layer. The coating layer is not limited to coating of the first polymer and the second polymer in the first and second layers, and even if only a single layer is coated by 5% or less, the same immediate release pattern effect is exhibited.

The lubricant improves the fluidity of ilaprazole particles, prevents friction between particles, and keeps the ilaprazole particles from adhering to a tableting machine. The lubricant may be one or more lubricants selected from the group consisting of magnesium stearate, stearic acid, zinc stearate, calcium stearate, talc, sodium stearyl fumarate, talc, silicon dioxide, and colloidal silicon dioxide, but is not limited thereto.

The fluidizing agent may play a role in preventing tablet components from adhering to the surface of a compressing machine and improving a flow rate of tablet granules so as to facilitate tablet compression. The fluidizing agent may be one or more fluidizing agents selected from the group consisting of talc, magnesium stearate, calcium stearate, stearic acid, glyceryl behenate, and polyethylene glycol, but is not limited thereto. In the present disclosure, the tablet may be manufactured via a dry direct compressing method in the manufacture, but is not limited thereto.

Therefore, the oral immediate-release formulation according to the present disclosure stabilizes the ilaprazole tablet, which is highly unstable under acidic conditions, using calcium hydroxide or magnesium hydroxide, thereby preventing decomposition of the active ingredient under acidic conditions and showing an effect of maintaining the storage stability of the formulation for a long period of time even under accelerated storage conditions. In addition, it may be widely used as an oral immediate-release nucleated tablet by making the ilaprazole rapidly absorbed in the stomach through the gastric acid neutralizing effect by sodium bicarbonate.

### MODES FOR CARRYING OUT THE INVENTION

Hereinafter, the present disclosure will be described in more detail through examples. These examples are only intended to describe the present disclosure in more detail, and it will be apparent to those skilled in the art that the scope of the present disclosure is not limited by these examples according to the gist of the present disclosure.

### Example 1. Evaluation of Formulation Stability According to a Ratio of Coating Agent in a Cored Tablet

As shown in Table 1 below, cored tablets of ilaprazole were coated using different ratios of a coating agent. A first coating solution containing HPMC as a coating agent and a second coating solution containing ethyl cellulose were used.

Each prepared tablet was stored for 2 and 4 weeks under room temperature, accelerated, and harsh storage conditions, and then the formulation stability was evaluated. The results are shown in Table 1 below.

TABLE 1

| Ingredients [% (w/w)] | | 1-1 | 1-2 | 1-3 |
|---|---|---|---|---|
| Ilaprazole | | 6.7% | 6.7% | 6.7% |
| D-Mannitol | | 30.7% | 30.7% | 30.7% |
| Hydroxypropyl cellulose | | 2.0% | 2.0% | 2.0% |
| Magnesium hydroxide | | 40.0% | 40.0% | 40.0% |
| Sodium starch glycolate | | 15.0% | 15.0% | 15.0% |
| Low-substituted hydroxypropyl | | 4.7% | 4.7% | 4.7% |
| cellulose | | | | |
| Magnesium stearate | | 1.0% | 1.0% | 1.0% |
| Cored tablet % | | 100.0% | 100.0% | 100.0% |
| Coating | First layer coating: | — | 1.00% | 1.00% |
| rate | HPMC | | | |
| | Second layer coating: | — | 2.00% | 5.00% |
| | ethyl cellulose | | | |
| Initial related substance | | 0.33% | 0.28% | 0.26% |
| Room | 2-week related substance | 0.42% | 0.26% | 0.25% |
| temper- | 4-week related substance | 0.83% | 0.26% | 0.27% |
| ature | | | | |
| Acceler- | 2-week related substance | 0.60% | 0.41% | 0.38% |
| ation | 4-week related substance | 1.01% | 0.52% | 0.50% |
| Harsh | 2-week related substance | 0.70% | 0.53% | 0.49% |
| | 4-week related substance | 1.68% | 0.71% | 0.66% |

The pharmaceutical composition of the present disclosure may be manufactured according to a conventional tablet manufacturing method. For example, using a tablet obtained by undergoing first coating with an HPMC-based coating agent included in the composition of the present disclosure and then second coating with an ethyl cellulose-based coating agent, the tablet compressed into a nucleated tablet including sodium bicarbonate as the outer layer is used as a composition of the present disclosure (or a nucleated tablet, in which contact between the main ingredients is suppressed by coating on the inner core using a coating agent including HPMC and ethyl cellulose, is used as the final composition).

From the results in Table 1 above, it is noticed that the total related substance of ilaprazole significantly increased in the uncoated tablets. In contrast, according to the present disclosure, when 2 to 5 wt % of the second coating agent was used with respect to 100 wt % of the entire inner core, it was found that the total amount of related substances generated from liarozole was significantly reduced, and there was almost no difference in the total amount of related substances generated between the two. Therefore, it was determined that the ratio of the second coating agent in the nucleated tablet of the present disclosure is preferable within 5 wt % with respect to 100 wt % of the entire inner core.

### Example 2. Evaluation of Intragastric pH Stability of Ilaprazole According to a Dose of Sodium Bicarbonate

As shown in Table 2 below, the stability of ilaprazole according to a dose of sodium bicarbonate was identified in a pH 4.0 buffer (postprandial gastric pH 3.5 to 5.0). Sodium bicarbonate was dissolved in various doses in the prepared pH 4.0 buffer, and 10 mg of ilaprazole was administered to observe the total amount of related substances generated from ilaprazole over time.

TABLE 2

| | Sodium | Total related substance (%) | |
|---|---|---|---|
| | bicarbonate | Content of ilaprazole | Total related substances |
| Buffer | (mg) | (%) | of ilaprazole (%) |
| pH 4.0 | 300 | 46.59 | 0.61 |
| 100 mL | 400 | 71.98 | 0.35 |
| | 500 | 77.90 | 0.34 |
| | 600 | 91.27 | 0.22 |
| | 700 | 100.65 | 0.14 |
| | 800 | 101.84 | 0.09 |
| | 900 | 100.19 | 0.11 |
| | 1000 | 100.27 | 0.10 |
| | 1100 | 99.95 | 0.09 |

From the results from the related substance test in Table 2 above, the total related substance generated from ilaprazole decreased drastically when sodium bicarbonate was in the range of 600 mg or greater, but in other ranges of sodium bicarbonate, excess related substances were generated from the unknown related substances among the standards (total related substances of 2.5% or less, known related substances of 0.5% or less, and unknown related substances of 0.2% or less). Therefore, through the above results, it was found that it is desirable to include 600 to 1,100 mg of sodium bicarbonate per nucleated tablet.

### Example 3. Six-Month Stability Evaluation of the Cored Tablet According to Stabilizers

As shown in Table 3 below, ilaprazole cored tablets were prepared in the same manner as in Example 1 using calcium hydroxide and sodium carbonate as stabilizers, depending on the range of calcium hydroxide and sodium carbonate with respect to the total composition. The prepared cored tablets were stored at room temperature and under accelerated storage conditions for 2 and 4 weeks, and the pharmaceutical stability of ilaprazole was measured. The results are shown in Table 3 below.

TABLE 3

| Purpose of | Ingredients | | | | | | | |
|---|---|---|---|---|---|---|---|---|
| compounding | [% (w/w)] | 3-1 | 3-2 | 3-3 | 3-4 | 3-5 | 3-6 | 3-7 |
| Main | Ilaprazole | 6.7% | 6.7% | 6.7% | 6.7% | 6.7% | 6.7% | 6.7% |
| ingredient | | | | | | | | |
| Excipient | D-Mannitol | 70.7% | 30.7% | 30.7% | 30.7% | 30.7% | 55.7% | 55.7% |
| Binder | Hydroxypropyl | 2.0% | 2.0% | 2.0% | 2.0% | 2.0% | 2.0% | 2.0% |
| | cellulose | | | | | | | |
| Stabilizer | Calcium | — | 40.0% | — | — | — | 15.0% | — |
| | hydroxide | | | | | | | |
| | Magnesium | — | — | 40.0% | — | — | — | 15.0% |
| | hydroxide | | | | | | | |
| | Sodium | — | — | — | 40.0% | — | — | — |
| | hydroxide | | | | | | | |
| | Sodium | — | — | — | — | 40.0% | | |
| | carbonate | | | | | | | |
| Disintegrant | Sodium | 15.0% | 15.0% | 15.0% | 15.0% | 15.0% | 15.0% | 15.0% |
| | starch | | | | | | | |
| | glycolate | | | | | | | |
| Binder | Low- | 4.6% | 4.6% | 4.6% | 4.6% | 4.6% | 4.6% | 4.6% |
| | substituted | | | | | | | |
| | hydroxypropyl | | | | | | | |
| | cellulose | | | | | | | |
| Lubricant | Magnesium | 1.0% | 1.0% | 1.0% | 1.0% | 1.0% | 1.0% | 1.0% |
| | stearate | | | | | | | |
| | % Total | 100.0% | 100.0% | 100.0% | 100.0% | 100.0% | 100.0% | 100.0% |
| Initial related substance | | 0.10% | 0.11% | 0.10% | 0.13% | 0.09% | 0.11% | 0.11% |
| Room | 2 M related | 0.49% | 0.20% | 0.19% | 0.24% | 0.36% | 0.23% | 0.27% |
| temperature | substance | | | | | | | |
| | 6 M related | 1.94% | 0.29% | 0.33% | 0.42% | 0.88% | 0.35% | 0.36% |
| | substance | | | | | | | |
| Acceleration | 2 M related | 5.01% | 1.02% | 0.99% | 1.35% | 1.99% | 1.14% | 1.20% |
| | substance | | | | | | | |
| | 6 M related | 9.43% | 2.64% | 2.67% | 3.17% | 3.98% | 3.09% | 3.08% |
| | substance | | | | | | | |

From the results in Table 3 above, it can be found that when calcium hydroxide and magnesium hydroxide were used as stabilizers in amounts of 15 to 40 wt % with respect to 100 wt % of the total cored tablets, the relative increase in the related substance (0%) of ilaprazole was minimal at both room temperature and an accelerated environment. Thereby, it was determined that ilaprazole had excellent stability in various environments when calcium hydroxide or magnesium hydroxide, which is a stabilizer, was contained in an amount of 15 to 40 wt % with respect to 100 wt % of the total cored tablet.

### Example 4. Evaluation of the Elution Rate Depending on Coating of Ilaprazole Tablets

An in vitro elution test was performed on nucleated tablets (nucleated tablets according to the coating agent ratio of the nucleated tablet) prepared in the same manner as in Example 1. After administering the prepared nucleated tablet, the elution solution was collected 0.083, 0.16, 0.25, 0.5, 0.75, and 1 hour later, and the content (%) of ilaprazole in the elution solution was analyzed. The resulting values are shown in Table 4 below.

TABLE 4

| Ingredients [% (w/w)] | | 1-1 | 1-2 | 1-3 |
|---|---|---|---|---|
| Ilaprazole (main ingredient) | | 6.7% | 6.7% | 6.7% |
| D-Mannitol | | 30.7% | 30.7% | 30.7% |
| Hydroxypropyl cellulose | | 2.0% | 2.0% | 2.0% |
| Magnesium hydroxide | | 40.0% | 40.0% | 40.0% |
| Sodium starch glycolate | | 15.0% | 15.0% | 15.0% |
| Low-substituted hydroxypropyl | | 4.7% | 4.7% | 4.7% |
| cellulose | | | | |
| Magnesium stearate | | 1.0% | 1.0% | 1.0% |
| Cored tablet % | | 100.0% | 100.0% | 100.0% |
| Coating rate | First layer coating: | — | 1.00% | 1.00% |
| | HPMC | | | |
| | Second layer coating: | — | 2.00% | 5.00% |
| | ethyl cellulose | | | |
| Main | 1 | 0.00 | 0.00 | 0.00 |
| ingredient | 5 | 71.87 | 29.61 | 22.89 |
| elution rate | 10 | 87.23 | 68.39 | 59.10 |
| over time | 15 | 93.15 | 84.11 | 83.78 |
| (%) | 30 | 98.96 | 93.84 | 95.21 |
| | 45 | 100.11 | 96.36 | 96.98 |
| | 60 | 101.95 | 97.35 | 98.56 |

From the above Example 1, an immediate-release nucleated tablet of ilaprazole and sodium bicarbonate was prepared. From the results in Table 4 above, when comparing the initial elution profiles according to different coating ratios of the cored tablet, it was found that the elution rate of the uncoated tablet was approximately three times faster than that of the coated tablet, although the ratio of the main ingredient was the same. However, according to the results of Example 1 above, it was found that the total amount of related substances in the uncoated tablets increased relatively by about twice as much as in the coated tablets. When the ratio of the coating agent containing ethyl cellulose was 5 wt % or less with respect to 100 wt % of the entire inner core, the elution rates were almost the same when compared.

In particular, the nucleated tablet prepared as “Preparation Example 1-2” in Table 4 above is physicochemically stable for a long period of time, and when administered orally, it exhibits an immediate-release form in the elution profile, such that it may be expected to be an immediate-release nucleated tablet that is stable and rapidly absorbed even in the gastric environment.

From the above description, those skilled in the art will understand that the present disclosure may be implemented in other specific forms without altering the technical idea or essential characteristics thereof. In this regard, it should be understood that the embodiments described above are merely illustrative in all respects, but not limiting. The scope of the present disclosure should be interpreted to include all changes or modified forms derived from the meaning and scope of the appended claims and their equivalent concepts in the scope of the present disclosure, rather than the detailed description above.

1. An oral immediate-release formulation comprising:
an inner core comprising ilaprazole as an active ingredient and a stabilizer; and
an outer layer surrounding the inner core and comprising an antacid.

2. The oral immediate-release formulation of claim 1, wherein the ilaprazole is included in an amount of 5 to 15 mg per formulation.

3. The oral immediate-release formulation of claim 1, wherein the stabilizer comprises one or more selected from the group consisting of calcium hydroxide, magnesium hydroxide, sodium hydroxide, meglumine, magnesium oxide, calcium carbonate, sodium bicarbonate, calcium hydrogen phosphate, potassium hydrogen phosphate dihydrate, calcium phosphate tribasic, monoethanolamine, potassium bicarbonate, potassium citrate, sodium borate, sodium citrate dihydrate, and triethanolamine.

4. The oral immediate-release formulation of claim 3, wherein the stabilizer is included in an amount of 15 to 40 wt % with respect to 100 wt % of the entire inner core.

5. The oral immediate-release formulation of claim 1, wherein the antacid comprises one or more selected from the group consisting of sodium bicarbonate, sodium carbonate, potassium carbonate, sodium glycine carbonate, calcium carbonate, sodium sulfite, sodium bisulfite, sodium metabisulfite, and calcium bicarbonate.

6. The oral immediate-release formulation of claim 5, wherein the antacid is included in an amount of 600 to 1100 mg per formulation.

7. The oral immediate-release formulation of claim 1, further comprising a coating layer containing a polymer between the inner core and the outer layer.

8. An oral immediate-release formulation comprising:
an inner core which comprises a tablet comprising ilaprazole as an active ingredient and calcium hydroxide as a stabilizer, and a polymer-containing coating layer coating the tablet; and
an outer layer which surrounds the inner core and comprises sodium bicarbonate as an antacid.

9. The oral immediate-release formulation of claim 7, wherein the polymer further comprises one or more polymers selected from the group consisting of hydroxypropyl methyl cellulose (HPMC), hydroxyethyl methyl cellulose, hydroxypropyl cellulose, hydroxymethyl cellulose, hydroxyethyl cellulose, carboxymethyl cellulose, carboxymethyl cellulose calcium, carboxymethyl cellulose sodium, ethyl cellulose, Eudragit L 100-55, a mixture of Eudragit RS and Eudragit RL, cellulose acetate, ethyl cellulose, cellulose acetate phthalate, hydroxypropyl methyl cellulose acetate succinate, hydroxypropyl methyl cellulose phthalate, polyethyl acetate-methyl acetate copolymer, polyvinyl phthalate acetate, and shellac.

10. The oral immediate-release formulation of claim 8, wherein the polymer included in the coating layer is included in an amount of 0.5 to 5 parts by weight with respect to 100 parts by weight of the entire inner core.

11. The oral immediate-release formulation of claim 1, wherein the outer layer further comprises povidone or copovidone as a binder.

12. The oral immediate-release formulation of claim 1, wherein the outer layer further comprises crospovidone or sodium starch glycolate as a dissolution aid.

13. The oral immediate-release formulation of claim 1, wherein, when the oral immediate-release formulation is administered orally, immediately after an antacid effect due to rapid elution of the antacid from the outer layer in the stomach, ilaprazole in the inner core is eluted.

14. The oral immediate-release formulation of claim 1, wherein, when a drug elution test is performed with 900 mL of water (Korean Pharmacopoeia, Elution Test Method) at a rotation rate of 75 rpm and a medium temperature of 37.5° C., an elution rate of the antacid at 15 minutes is 80% or greater and that at 30 minutes is 100% or greater.

15. The oral immediate-release formulation of claim 13, wherein the antacid is sodium bicarbonate.

16. The oral immediate-release formulation of claim 1, wherein, when the oral immediate-release formulation is administered to 500 mL of pH 1.2 buffer, and then, after approximately 10 minutes which is the time for the outer layer to almost disintegrate or be eluted, additional elution solution to make 900 mL (final volume of 900 mL) of pH 10.0 buffer (FDA dissolution method DB, Rabeprazole sodium Tablets) is added to conduct a drug elution test at a rotation rate of 100 rpm and a medium temperature of 37.5° C., the elution rate of ilaprazole at 30 minutes is 80% or greater.

17. A method of preparing an oral immediate-release formulation, the method comprising:
(i) mixing ilaprazole, a stabilizer, and a pharmaceutically acceptable additive to prepare a mixture (step 1);
(ii) first compressing the mixture to prepare an inner core in the form of a tablet (step 2); and
(iii) second compressing a mixture obtained by mixing an antacid and a pharmaceutically acceptable additive on the inner core to prepare an outer layer in the form of a tablet (step 3).

18. The method of claim 17, further comprising preparing a coating layer containing a polymer between the second and third steps.

19. A method of treating an acid-related gastrointestinal disorder in a subject in need thereof, comprising:
orally administering to the subject the oral immediate-release formulation of claim 1.

20. A method of treating an acid-related gastrointestinal disorder in a subject in need thereof, comprising:
orally administering to the subject the oral immediate-release formulation of claim 8.
